(19) United States
(12) Patent Application Publication
(10) Pub. No.: US 2009/0163976 A1
(43) Pub. Date: Jun. 25, 2009

(54) Methods and Devices for the Study and Treatment of Surgical and Chronic Pain with Transcranial Magnetic Stimulation (TMS)

(76) Inventors: Jeffrey Borckardt, Charleston, SC (US); Scott Reeves, Mt. Pleasant, SC (US); Mark George, Sullivans Island, SC (US); John Walker, Mt. Pleasant, SC (US)

Correspondence Address: MUSC FOUNDATION FOR RESEARCH DEVELOPMENT, 19 HAGOOD AVE, SUITE 909, P.O. BOX 250828, CHARLESTON, SC, 29425, US

(21) Appl. No.: 12/197,400

(22) Filed: Aug. 25, 2008

Related U.S. Application Data
(60) Provisional application No. 60/957,844, filed on Aug. 24, 2007.

(51) Int. Cl. A61N 1/36

(52) U.S. Cl. 607/46

(57) ABSTRACT

Embodiments of this invention comprise devices and methods for the treatment of acute surgical pain and chronic pain syndrome through the use of rTMS. Further embodiments comprise a sham TMS system for use in clinical research.

This application claims benefit of priority to U.S. Provisional No. 60/957,844 filed on Aug. 24, 2007, entitled “Methods and Devices for the Treatment of Surgical and Chronic Pain with Transcranial Magnetic Stimulation (TMS)” which is incorporated herein by reference in its entirety.

### BACKGROUND OF THE INVENTION

Transcranial magnetic stimulation (TMS) employs an electro-magnet placed externally over the scalp. This electro-magnet, often called a coil, generates magnetic field pulses which can pass through the outer skin and skull into the underlying tissues of the brain. The magnetic field passes unimpeded through the skin and skull, inducing an oppositely directed current in the brain that flows tangentially with respect to the skull. The current induced in the structure of the brain activates nearby nerve cells in much the same way as currents applied directly to the cortical surface. Various treatment modalities are possible using TMS.

### BRIEF SUMMARY OF THE INVENTION

Embodiments of this invention comprise devices and methods for the use of TMS to treat acute surgical pain. Additional embodiments of this invention comprise devices and methods for the use of TMS to treat chronic pain syndromes. More specifically, certain embodiments utilize repetitive transcranial magnetic stimulation (rTMS) for the treatment of acute surgical pain or chronic pain syndromes. Embodiments of the invention comprise a rTMS device for treating acute surgical pain wherein the parameters comprise 10 Hz, 100% rMT, 10 secs ON, 20 secs OFF, for 20 minutes over the left-prefrontal cortex.

### BRIEF DESCRIPTION OF THE DRAWINGS

FIG. 1: Change in thermal pain threshold (measured via method of limits using a TSA-II Thermosensory Analyzer) following 15 minutes of either active or sham left-prefrontal rTMS in healthy adults (N=12).

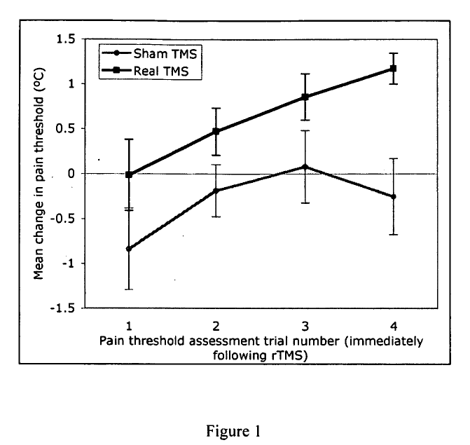

FIG. 2: Mean cumulative patient controlled analgesia (PCA) pump usage in mg of morphine for patients randomly assigned to 20 minutes (4000 pulses) of either active or sham left-prefrontal TMS following gastric bypass surgery (N=20).

FIG. 3: Mean (and standard error) absolute PCA-delivered morphine used per 8 hours by patients receiving either real or sham TMS after gastric-bypass surgery (N=20).

FIG. 4: MS delivery in the Post-Anesthesia Recovery Unit (PACU). The picture depicts the resting Motor Threshold (rMT) assessment procedure (which involves stimulation of the motor cortex). Custom-developed software was used to run a Parameter-Estimation by Sequential Testing (PEST) algorithm, and visible thumb movement (APB) was used to determine the amount TMS machine output necessary to stimulate the cortex.

FIG. 5: Screen shot of the software used to collect sensation ratings and to assess the areas of the face and scalp where the sensations were felt.

FIG. 6: Mean (and 95% CI) visual analogue scale ratings for each of the sensory dimensions assessed during real and active-sham TMS.

FIG. 7: Mean face and scalp areas of activation during both real and active-sham TMS

### DETAILED DESCRIPTION OF THE INVENTION

Background: Chronic pain is a large public health concern affecting millions of Americans and resulting in billions of dollars per year in direct and indirect healthcare costs, lost-wages and disability expenses. Pain is a complex experience that has sensory-discriminatory, motivational-affective and cognitive-evaluative dimensions and depression prevalence rates in patients with persistent pain range from 30%-54% when rigorous criteria are used to diagnose depression. Chronic motor cortex stimulation (MCS) via implanted electrodes has been used to achieve pain control in patients with intractable neuropathic pain. However, the mechanisms by which MCS controls pain are unclear. Some research suggests that MCS may work by impacting the cognitive and affective components of pain experience. Unfortunately, MCS is an invasive and expensive surgical procedure.

### Example 1

Methods: In the current studies, we investigated the effects of left prefrontal rTMS on controlled thermal laboratory pain in healthy adults and on post-operative pain (measured by Patient Controlled Analgesia (PCA) pump usage) in gastric bypass surgery patients. Laboratory thermal pain procedures were used to assess pain thresholds and suprathreshold pain estimates (visual analog scale; VAS) in 12 healthy adults pre and post 15 minutes of either active or sham prefrontal TMS (using an array device parameters). In the second study, 20 gastric bypass surgery patients received left prefrontal rTMS immediately following surgery (100% of resting motor threshold, 10 Hz, 10-second stimulus train, 20-second interstimulus interval for 20 minutes; a total of 4000 pulses). Patients were randomly assigned to receive either active or sham TMS and were blind to condition. PCA pump usage was tracked during the 2 days following surgery. A third study investigating the effects of prefrontal rTMS on neuropathic pain is currently in progress.

Results: In the healthy adult cohort, 39% of subjects that received active prefrontal TMS exhibited a decrease in thermal pain ratings compared to 17% in the sham TMS condition. Additionally, a trend toward increasing thermal pain thresholds was observed after active rTMS. In the gastric-bypass surgery cohort, a significant effect for active prefrontal TMS was observed on PCA pump usage. Subjects that received active TMS used 40% less total morphine than subjects receiving sham TMS. The effect was most observable during the first 24-hours following TMS. Results from the neuropathic pain study are not yet available.

Discussion: rTMS can be a treatment for certain chronic pain conditions (especially neuropathic pain and in patients with co-morbid depression).

### Example 2

Background: Several recent studies suggest that repetitive transcranial magnetic stimulation can temporarily reduce pain perception in neuropathic pain patients and in healthy adults using laboratory pain models. No studies have investigated the effects of prefrontal cortex stimulation using transcranial magnetic stimulation on postoperative pain.

Methods: Twenty gastric bypass surgery patients were randomly assigned to receive 20 min of either active or sham left prefrontal repetitive transcranial magnetic stimulation immediately after surgery. Patient-controlled analgesia pump use was tracked, and patients also rated pain and mood twice per day using visual analog scales.

Results: Groups were similar at baseline in terms of body mass index, age, mood ratings, pain ratings, surgery duration, time under anesthesia, and surgical anesthesia methods. Significant effects were observed for surgery type (open vs. laparoscopic) and condition (active vs. sham transcranial magnetic stimulation) on the cumulative amount of patient-delivered morphine during the 44 h after surgery. Active prefrontal repetitive transcranial magnetic stimulation was associated with a 40% reduction in total morphine use compared with sham during the 44 h after surgery. The effect seemed to be most prominent during the first 24 h after cortical stimulation delivery. No effects were observed for repetitive transcranial magnetic stimulation on mood ratings.

Conclusions: A single session of postoperative prefrontal repetitive transcranial magnetic stimulation was associated with a reduction in patient-controlled analgesia pump use in gastric bypass surgery patients. This is important because the risks associated with postoperative morphine use are high, especially among obese patients who frequently have obstructive sleep apnea, right ventricular dysfunction, and pulmonary hypertension.

Detailed Discussion:

For many years, it has been known that chronic motor cortex stimulation (MCS) via implanted epidural electrodes controls neuropathic pain.¹⁻⁴ The antinociceptive mechanisms of MCS are unclear; however, the magnitude of pain relief is correlated with activation of portions of the anterior cingulate and orbitofrontal cortex.^{5,6} Thus, MCS may exert some of its analgesic effect by altering the affective dimension of pain experience.

Transcranial magnetic stimulation (TMS) is a noninvasive brain stimulation technology that can focally stimulate the brain of an awake individual.^{7,8} A localized pulsed magnetic field transmitted through a figure-eight coil induces electrical currents in the brain⁹ and focally stimulates the cortex by depolarizing superficial neurons.^{10,11} TMS at different intensities, frequencies, and coil angles excites several elements (e.g., cell bodies, axons) of various neuronal groups (e.g., interneurons, neurons projecting into other cortical areas).¹²⁻¹⁴ When TMS pulses are delivered repeatedly, it is referred to as repetitive transcranial magnetic stimulation (rTMS).

Findings from studies of rTMS for depression and from studies that integrate TMS and functional magnetic resonance imaging suggest that TMS over the prefrontal cortex can cause secondary activation in important pain and mood-regulating regions, such as the cingulate gyrus, orbitofrontal cortex, insula, and hippocampus.¹⁵ Moreover, rTMS affects the perception of laboratory-induced pain in healthy adults as well as chronic neuropathic pain in clinical samples. ¹⁶⁻²⁹ Although most of these investigations have shown short-lived effects of rTMS on pain, a recent study demonstrated that antinociceptive effects can be sustained for at least 15 days after 3 consecutive days of rTMS.²⁸

Following the literature from MCS, most studies of rTMS effects on pain perception have targeted the motor cortex. This approach is frequently hypothesized to work by normalizing activity of sensory neurons corresponding with the painful area. ^{20,24,27} However, as noted previously, much of the variance in clinical response to MCS seems to be explained by limbic activity.^{5,6} If one of the mechanisms by which cortical stimulation alleviates pain is by modulating the processing of the affective dimension of pain experience, the prefrontal cortex might be a more efficient cortical target for pain management.¹⁵ Consistent with this notion, a few recent studies have demonstrated acute and transient antinociceptive effects with prefrontal cortex TMS.^{23,29,30} In addition, functional imaging research has shown that activation of the left dorsolateral prefrontal cortex is associated with decreases in pain unpleasantness ratings in healthy adults using laboratory pain induction methods, and it has been proposed that the left prefrontal cortex may inhibit limbic activity associated with painful stimuli.³¹

Although a number of studies have been conducted on the effects of rTMS on chronic neuropathic pain, none to date have investigated the effects of rTMS on acute postoperative pain. This is an important area of study because postsurgical pain is associated with high levels of opioid medication use, and both the immediate and longer-term risks associated with these medications are high, especially among gastric bypass surgery patients who frequently have obstructive sleep apnea, right ventricular dysfunction, and pulmonary hypertension. Therefore, we conducted this study to assess whether one session of prefrontal rTMS could reduce postoperative pain and patient-controlled analgesia (PCA) pump use. In addition, because left prefrontal rTMS has been associated with improvements in mood,^{32,33} and mood has been shown to impact pain experience,³⁴ we examined the effects of TMS on post-TMS mood ratings (visual analog scale).

Materials and Methods

Despite little published information on the effect size for rTMS on pain perception, we arrived at a rough estimate of effect size based on tables and figures from the available rTMS/pain literature (mean Cohen d=1.32). To reach minimum acceptable power (0.80) for pairwise comparisons with an effect size of 1.32, 8 subjects were needed in each group. To minimize the probability of making a type II error for this pilot trial, 10 subjects were recruited for each group, which improved the estimated power to 0.88.

Twenty gastric bypass surgery patients were enrolled (mean age, 43.05 yr; mean body mass index, 50.27 kg/m2; 19 female). Gastric bypass was chosen for the surgical procedure in this first test of the postoperative antinociceptive effects of TMS because of the homogeneity of the patient population and because it afforded the opportunity to evaluate open and laparoscopic surgical procedures on a similar patient population. This study was approved by the Institutional Review Board for Human Research at the Medical University of South Carolina (Charleston, S.C.). Written informed consent to participate was obtained before laparoscopic (n=12) or open gastric bypass surgery (n=8). Open gastric bypass surgery was used in patients with previous abdominal surgery or a body mass index greater than 60 kg/m2. Intraoperative management consisted of intravenous premedication with midazolam and induction with propofol, lidocaine, fentanyl, and succinylcholine. Maintenance of anesthesia was accomplished with desflurane, fentanyl (up to 5 μg/kg), and cisatracurium. Reversal of neuromuscular blockade was performed with neostigmine and glycopyrrolate. All patients were pretreated for postoperative nausea and vomiting with ondensetron 30 min before emergence from surgery. At the time of arrival in the recovery room, patients were loaded with morphine sulfate up to 0.1 mg/kg ideal body weight based on their clinical level of assessed pain. This individual titration was performed by nursing staff who were blinded to the study protocol and patient randomization scheme.

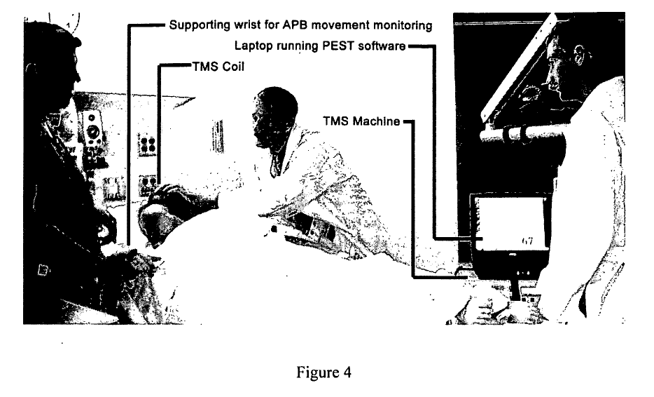

After surgery, in the postanesthesia care unit, each subject underwent a motor threshold assessment. The TMS device was set to 80% of machine output and fired single pulses at the rate of 1 per 2 s (0.5 Hz). The coil was systematically moved around the left scalp, and the stimulus intensity was adjusted until the area of the motor cortex involved in movement of the right abductor pollicis brevis was located. The interstimulus interval was then decreased to 4 s (0.25 Hz), and a custom-designed software program was used to run an adaptive Parameter Estimation by Sequential Testing (PEST) algorithm.³¹ The researchers, with the aid of the program, determined the amount of TMS machine output necessary to produce visibly detectable movement of the thumb 50% of the time (resting motor threshold).^{35,36} Subjects' prefrontal cortices were then located by moving the coil 5 cm anterior from the area of the motor cortex associated with thumb movement along the parasagittal line. FIG. 4 shows the TMS setup in the postanesthesia care unit during a resting motor threshold assessment.

After the motor threshold assessment, subjects were started on a morphine PCA pump. The PCA pump was set at a 1-mg bolus with a 6-min lockout interval. If the patient was allergic to or could not tolerate morphine sulfate, hydromorphone was used instead (n=2; 1 in the sham group, 1 in the active group). For statistical analyses of PCA pump use, morphine equivalent analgesic dose was calculated (0.2 mg hydromorphone=1.0 mg morphine).

Subjects were randomly assigned to receive real rTMS (n=10) or sham rTMS (n=10). Randomization was accomplished with the aid of a custom-developed visual BASIC application that was designed to randomize the TMS condition (real vs. sham) within the constraints of the predetermined group sizes (a total of 12 laparoscopic and 8 open surgery cases) and to ensure equal numbers of patients in the groups (10 active and 10 sham). Thus, there were 6 laparoscopic and 4 open surgery cases in each group. The randomization scheme was stored in a spreadsheet file that was available only to the researcher who was responsible for delivering rTMS. Contact between this researcher and the subject was limited to the motor threshold assessment and TMS delivery session. The researcher was not involved in the data collection process.

The active TMS coil is a figure-eight design with a solid core interior (Neopulse Neotonus devices, Malvern, Pa.). The sham coil is externally identical to the active TMS coil except that it does not actually stimulate, because an aluminum insert on the surface next to the scalp blocks passage of the magnetic field. Subjects received 20 min of 10-Hz rTMS at 100% of resting motor threshold (10-s stimulation trains with 20-s interstimulus intervals) for a total of 4,000 pulses. This dose is within the published safety guidelines,³⁷ although it is higher than most used in previously published studies on the effects of rTMS on pain perception. However, most other TMS/pain studies have examined the effects of motor cortex stimulation on pain perception. The motor cortex is more excitable than prefrontal cortex.^{37,38} Therefore, a higher dose may be necessary to achieve desired effects when targeting the prefrontal cortex. In addition, the investigators were interested in maximizing the potential effects of rTMS on pain perception and did not want to err on the side of underdosing and risk a type II error during this pilot trial. This dosing decision was, of course, balanced against potential risks to the patients, which were determined to be minimal given that (1) the dose conformed to the published safety guidelines, (2) the cortical target (prefrontal cortex) is less excitable and is associated with a lower risk for seizures than the motor cortex, and (3) in the postanesthesia care unit, there is immediate availability of highly skilled physicians and nursing staff as well as the availability of critical care equipment if a seizure were to occur.

Subjects provided visual analog scale ratings of mood twice per day (0=extremely sad or depressed and 100=extremely happy or great mood), and PCA pump use data were collected from each subject's medical record once per day (morphine use data were available in 2-h intervals). Subjects, medical staff providing clinical care to subjects, and personnel collecting ratings were blind to whether subjects had received real or sham rTMS. The only person who knew the randomization was the rTMS administrator (J.J.B.), who was not aware of the surgical history and medication loading and who followed a careful script with patients, physicians, and nurses.

Statistical Analysis

Independent sample t tests were used to compare the active and sham TMS groups across a number of baseline variables that might have influenced postoperative PCA pump use. Hierarchical linear modeling was used to assess the effects of surgery type (laparoscopic vs. open) and rTMS condition (real vs. sham) on cumulative PCA pump use curves over time. Hierarchical linear modeling has been shown to appropriately handle nested models with serially dependent data points,^{39,40} and it allows for modeling of variables at the individual subject level (e.g., each subject's cumulative PCA pump use over time) and at the broader organizational level to which each individual belongs or is assigned (e.g., surgery type and TMS condition). All subjects' PCA orders in this study were discontinued after 44 h. This time frame was clinically determined and was independent of the study protocol. Cumulative PCA use curves over 44 h after surgery were square-root transformed to correct for nonlinearity and nonnormality. The estimation method of the model was restricted maximum likelihood, and the covariance structure was “unstructured.” Means are reported with accompanying SE values. An independent sample t test was used to compare postoperative mood ratings between groups, and hierarchical linear modeling was used to assess for differences between groups in change in mood over time.

Results

No significant differences were found between the active and sham TMS groups in terms of pre-TMS pain ratings, pre-TMS mood ratings, surgery duration, anesthesia duration, morphine loading, or fentanyl, hydromorphone, ketorolac, or lidocaine use. A significant difference was found between groups for midazolam use (P=0.04). However, the active TMS group was given less midazolam (1.75 mg) than the sham group (3.0 mg), which, if anything, would be expected to reduce PCA pump use in the sham group. Table 1 shows the means and SEs (as well as P values from the independent t tests) for each of these variables for both the active and sham TMS groups.

TABLE 1
Means and SEs for Subject Characteristics and Key Variables before
(or Immediately after) TMS for Each Group (Active or Sham)

| | Active, Mean | | Sham, Mean | | |
|---|---|---|---|---|---|
| Variable | (SEM) | | (SEM) | | P Value |
| Age, yr Pre-TMS pain, | 45.60 | (3.28) | 40.50 | (2.86) | 0.56 |
| VAS Post-TMS pain, | 61.00 | (10.26) | 64.50 | (7.98) | 0.79 |
| VAS Pre-TMS mood, | 58.90 | (6.30) | 57.60 | (5.35) | 0.88 |
| VAS Post-TMS mood, | 55.67 | (7.75) | 58.33 | (4.55) | 0.76 |
| VAS Body mass | 50.80 | (6.92) | 61.60 | (3.82) | 0.19 |
| index, kg/m2 Surgery | 49.01 | (2.16) | 51.54 | (4.17) | 0.60 |
| duration, min | 117.42 | (3.04) | 126.12 | (7.24) | 0.28 |
| Anesthesia duration, | 189.54 | (7.21) | 190.02 | (8.88) | 0.97 |
| min Midazolam, mg | 1.75 | (.37) | 3.00 | (.42) | 0.04 |
| Fentanyl, _g Pre-PCA | 277.50 | (34.65) | 317.50 | (39.62) | 0.46 |
| morphine, mg | 8.80 | (1.61) | 9.20 | (1.98) | 0.88 |
| Hydromorphone, mg | 0.20 | (.20) | 0.33 | (.33) | 0.84 |
| Ketorolac, mg | 12.00 | (4.90) | 20.00 | (4.47) | 0.24 |
| Lidocaine, mg | 84.44 | (8.01) | 90.00 | (6.15) | 0.56 |

No significant differences were found except for midazolam use; however, subjects in the sham transcranial magnetic stimulation (TMS) group were given more midazolam than subjects in the active TMS group. PCA = patient-controlled analgesia; VAS = visual analog scale score.

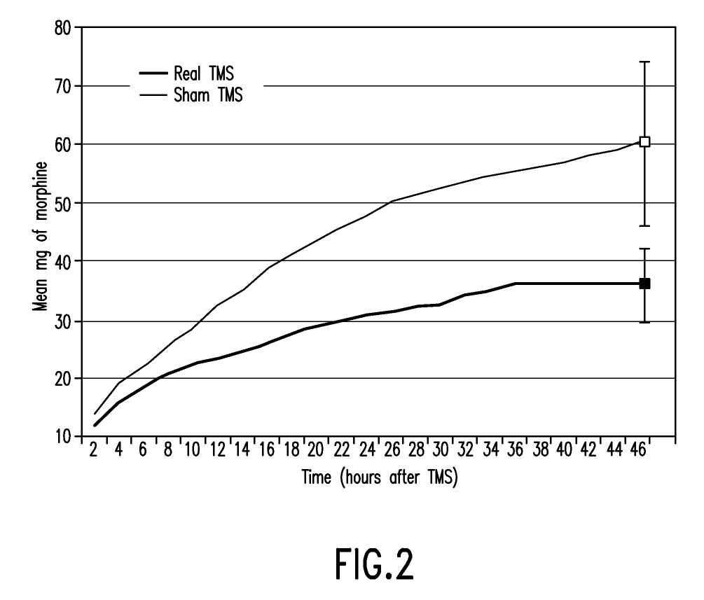

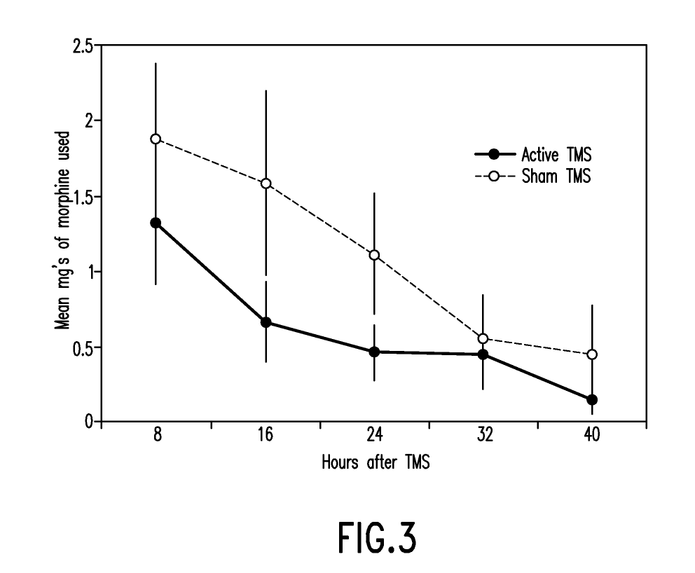

Significant effects were observed for both rTMS condition (t(436)=5.72, P<0.0001) and surgery type (t(436)=7.69, P<0.0001) on PCA pump use over time. Model estimates suggest that subjects receiving active rTMS used 1.21 (0.21) cumulative milligrams of morphine less than subjects in the sham condition per 2 h. FIG. 2 shows the mean cumulative morphine use for subjects in each group. At the time of discharge, subjects who had received real rTMS had used an average of 40% less morphine than subjects who had received sham rTMS. Subjects in the active rTMS group used 36.10 (6.27) mg on average, and subjects receiving sham rTMS used 60.18 (14.70) mg. FIG. 3 displays mean absolute morphine use in 8-h blocks after surgery for subjects in each group. The largest absolute difference between active and sham TMS seems to occur within the first 24 h after stimulation (determined by visual inspection of the figure). Model scores suggest that subjects receiving laparoscopic surgery used 1.48 (0.19) mg morphine less than subjects receiving open surgery per 2 h during the 44 h after surgery. At the time of discharge, subjects who received laparoscopic surgery had used an average of 34.90 (7.25) mg morphine, and subjects receiving open surgery used 68.00 (15.61) mg.

The average mood rating for subjects who received active rTMS was 78.24 (4.97), and the mean for subjects receiving sham was 72.33 (4.78). These mean ratings were not significantly different (t(18)=0.86). In addition, there were no effects observed for TMS condition on change in mood ratings over time (using hierarchical linear modeling; t(66)=1.32). At the time of discontinuation of the PCA pumps, the mean mood ratings of subjects who received active TMS was 73.11 (6.36), and the mean for subjects who received sham TMS was 74.33 (6.76). Therefore, in our sample, a single 20-min prefrontal TMS session did not seem to produce changes in mood ratings relative to sham TMS.

Throughout the study, two subjects in the active rTMS group (20%) reported nausea, as did two from the sham group (20%). No subjects in the study vomited during the hospital stay. However, 50% (n=5) of the subjects in the active rTMS group reported headache at some point during their hospital stay after rTMS, whereas only 20% (n=2) of the subjects in the sham group reported headache. Group assignment (real or sham) was not a significant predictor of headache status (Cox and Snell R 2=0.10; Wald=1.8; odds ratio=0.25, P=0.17). In all cases, the headaches were not severe and were easily managed using standard clinical pain protocols. No unusual measures were necessary for managing discomfort or complications in subjects who received active rTMS relative to those receiving sham.

Discussion

This trial indicates that a single 20-min postoperative prefrontal rTMS session in gastric bypass surgery patients may significantly reduce patient-administered morphine use over time. This effect seems to be most prominent during the first 24 h after rTMS delivery.

The mechanisms by which rTMS modulates pain experience are unclear. However, previous research suggests that rTMS may lead to inhibition of limbic activity associated with both pain and depressed mood. The findings from this study show that rTMS may be used to modulate pain experience during critical time periods to alter the course of acute pain and the consequent trajectory of opioid use. Previous research on TMS and pain experience suggests that multiple TMS sessions are needed to cause detectable changes in pain perception. However, it should be noted that the TMS dose used in this study was much higher than what previous studies have used. Embodiments include both single and multiple dosing treatments.

The effect of being on the real rTMS trajectory translated into an average decrease of 24.08 mg morphine at discharge (40%). This degree of morphine reduction is clinically significant in this group of patients who frequently have obstructive sleep apnea, right ventricular dysfunction, and pulmonary hypertension. Although thoracic epidurals may be used to reduce morphine use in many surgical patients, unfortunately they may be difficult to place in these morbidly obese patients.

There is no evidence to date that TMS is associated with respiratory depression. Although not specifically studied, it is possible that patients would experience a decrease in pulmonary complications in those who received rTMS. This possibility should be evaluated in future trials.

It is important to note that all patients were given morphine sulfate postoperatively by nursing staff before sham or active rTMS administration and initiation of the PCA pump. Although there was no significant difference between the two groups in terms of pre-TMS morphine administration, the active group was given slightly less than the sham group. This minimizes the likelihood that the observed difference in PCA pump use between groups could be a carryover effect from a higher baseline morphine loading. Consistent with previous research on the effects of rTMS on pain perception,²⁸⁻³⁰ the observed effect may have been somewhat short-lived (<24 h). This relatively acute and rapid effect, if validated, suggests that additional benefit and reduction of narcotic use may be observed if rTMS is repeated within the first 24 h after surgery.

Previous studies on the effects of rTMS on pain perception have focused on neuropathic pain in clinical samples,^{16,18,20,25,26,28} or on laboratory pain induced in healthy adults.^{21,22,24,29,30} Most of these studies have examined how motor cortex stimulation effects pain perception. There are only two published reports to date examining the effects of prefrontal rTMS on pain perception. One is a case report of a single subject with chronic pain, and the other is a laboratory study using healthy adults with slow right prefrontal TMS.²⁹ Both studies reported significant antinociceptive effects of TMS. The majority of published studies investigating the effects of rTMS on pain perception (clinical or laboratory) report promising, although short-lived results. The current study is the first to demonstrate the impact of appropriately timing a brief TMS intervention in a predictable acute pain scenario.

All subjects were attached to standard monitoring units (heart rate, pulse oximetry, blood pressure, and respiratory rate). There was minimal interference of the rTMS machine with these monitors and no observed heating of electrodes. Two subjects in the active rTMS group (20%) reported nausea, as did two from the sham group (20%). No subjects in the study vomited during the hospital stay. However, 50% (n=5) of the subjects in the active rTMS group reported headache at some point during their hospital stay after rTMS, whereas only 20% (n=2) of the subjects in the sham group reported headache. Group assignment (real vs. sham) was not a statistically significant predictor of headache status in our small sample, but there is some evidence that rTMS can cause headaches for some patients.³⁷ This risk is routinely presented to potential rTMS subjects during the informed consent process. In this study, none of the reported headaches were rated as severe by the subjects, and all were easily managed using standard clinical pain protocols. No unusual measures were necessary for managing discomfort or complications in subjects who received active rTMS relative to those receiving sham.

This trial is the first to demonstrate that a single 20-min prefrontal rTMS session in a postoperative setting can significantly reduce PCA morphine use.

### Example 3

Transcranial magnetic stimulation (TMS) is a noninvasive brain stimulation technology that can focally stimulate the cortex of an awake individual (George et al, 2003; Barker & Jalinous, 1985). TMS involves delivery of a pulsed magnetic field through a figure-8 coil which induces electrical currents in the brain (Barker, Freeston, Jarratt & Jalinous, 1989) focally stimulating the cortex by depolarizing superficial neurons (George & Belmaker, 2000; George, Lisanby & Sackeim, 1999). TMS at different intensities, frequencies and coil angles can excite several elements (e.g., cell bodies, axons) of various neuronal groups (e.g., interneurons, neurons projecting into other cortical areas; Roth, Saypol, Hallet & Cohen, 1991; Amassian, Eberle, Maccabee & Cracco, 1992; Davey, Cheng & Epstein, 1991).

Several studies have found that rTMS delivered over motor cortex can affect the perception of laboratory-induced pain in healthy adults as well as chronic neuropathic pain in clinical samples (Migita, Uozumi, Arita & Monden, 1995; Rollnik, Wustefeld, Dauper, Karst, Fink, Kossev & Dengler, 2002; Lefaucher, Drouot, Keravel & Nguyen, 2001; Topper, Hfoltys, Meister, Sparing & Boroojerdi, 2003; Pleger, Janssen, Shwenkreis et al, 2004; Tamura, Tatsuya, Oga et al, 2003; Summers, Johnson, Pridemore & Oberoi, 2004; Lefaucheur, Drouot, Menard-Lefaucher & Nguyen, 2004; Canavero, Bonicalzi, Dotta et al, 2002; Khedr, Kotb, Kamel, Ahmed, Sadek & Rothwell, 2005). Additionally, a few studies have demonstrated anti-nociceptive effects with TMS over the prefrontal cortex TMS (Borckardt et al, 2006; Reid and Pridmore, 2001; Graff-Guerrero, Gonzalez-Olivera, Fresan, Gomez-Martin, Mendez-Nunez & Pellicer, 2005; Sampson et al, 2006).

One-significant limitation of most of the research on the effects of TMS on pain perception to date concerns the nature of the placebo or sham conditions employed. When TMS pulses are delivered repetitively, it is often experienced as painful (and at a minimum it produces noticeable scalp and/or facial sensations). Most sham TMS techniques (whether they involve tilting the coil away from the scalp or whether a specially designed sham TMS coil is used) produce identical sounds to active TMS, but they do not cause any scalp or facial sensation or discomfort. This is a serious problem when investigators are attempting to evaluate the effects of TMS on pain perception because, arguably, the painfulness of real TMS may lead to changes in pain perception independent of the intended cortical stimulation. A typical TMS session lasts 20-minutes, and it is possible that the painfulness of the experience triggers pain modulatory activity in research subjects (e.g., endogenous opioid activity, cognitive changes, activation of other descending pain inhibitory mechanisms). Thus, when comparing the effects of real TMS to sham TMS on pain perception, any observed antinociceptive effects of real TMS may be simply due to exposing subjects to a 20-minute painful procedure. These effects may have little or nothing to due with changes in cortical activation. Until a simple, affordable sham TMS system is available that produces facial/scalp sensations comparable to real TMS, valid inferences about the effects of TMS on pain perception will be limited.

Given the previous research showing that TMS may have the potential to induce changes in pain perception, it is important to being to develop research technologies that can improve the quality of future TMS/pain research so that more definitive conclusions can be drawn about the effects of TMS on pain. We describe the development of a portable (and relatively inexpensive) sham TMS system designed to mimic real TMS with respect to perceived facial/scalp sensations, and painfulness. Next, we present data from a small pilot trial in which the sensations and location (scalp and/or facial) produced by the sham system are compared to those produced by real TMS.

Methods

Sham TMS System Development: In a current multi-site NIH sponsored trial of left prefrontal TMS for depression, the James Long sham TMS system is being employed. This system integrates a Mecta (specs) system with a Neuronetics (specs) TMS machine. Two electrodes from the Mecta system are placed on the subjects forehead anterior to the TMS coil and the Mecta system is attached to the Neuronectics TMS machine. Every time a sham TMS pulse is delivered, a TTL pulse is sent from the TMS machine to the Mecta triggering a brief, mild electrical pulse that is delivered through the electrodes to the subject's scalp. This system also employs an auditory masking system so that neither the subject or the TMS operator can hear the TMS pulses being delivered thereby reducing the chances of identifying whether real or sham TMS is being delivered by picking-up on very subtle differences in the sound of real versus sham TMS. The James Long system provides an extremely high quality method for conducting double-blind TMS trials. However, it is quite expensive and requires the use of a lot of bulky equipment (2 separate computers plus the Mecta machine and digital display).

With TMS research expanding into different hospital settings, there is a need for a portable, convincing sham TMS system. We sought to develop a system that would be light, portable, inexpensive and that produced scalp sensations similar to real TMS. A small electrical stimulus generator (powered by a 9-volt battery) was used to delivery a constant stimulus (150 pulses per second) to a custom developed switch-box (described below). Two ½-inch, round, metal electrodes are attached from the switch box to the subject's forehead immediately anterior to the TMS coil and held in place by a rubber strap. A BNC cable connects the TMS machine to the switch-box and every time the TMS machine delivers a pulse, a TTL signal is sent via the BNC cable to the switch-box. Upon receiving the TTL pulse, the switch box opens a gate for ˜250 as allowing a brief electrical stimulus through to the subject's scalp. Thus, the subjects experience a brief electrical pulse every time the sham TMS coil clicks. The intensity of the stimulus is adjustable at the electrical generator (1 to 60 mA) and the time that the gate is let open after each TTL trigger is adjustable on the switch-box.

### Subjects

Nine non-depressed adults (3 female) with no history of chronic pain disorders volunteered to participate in this study approved by the Institutional Review Board for the Protection of Human Subjects at the Medical University of South Carolina. All subjects were free of medications known to lower seizure threshold, had no implanted medical devices, and had no history of stroke or seizure.

### Motor Threshold Assessment and Coil Placement

After providing written informed consent, resting motor threshold was estimated. A Neotonous Neopulse TMS machine was set to 40% of maximum machine output. The TMS coil was positioned over each subject's motor cortex and pulses were delivered at the rate of 1 per 4 seconds. The intensity and location of the stimuli delivered were systematically adjusted until the area of the motor cortex that controls the Abductor Pollicus Brevis muscle (APB) was located. Next, a parameter estimation by sequential testing (PEST) algorithm was used to determine the amount of machine output necessary to produce visual thumb movement 50% of the time (resting motor threshold; rMT). After motor threshold was assessed, the prefrontal cortex was located by moving the coil 5 cm anterior along a parasagittal line. The coil position was marked on the subject's scalp using a non-toxic felt-tipped marker.

### Titrating the Sham TMS System:

Next, the electrodes from the portable sham system were attached to each subject's forehead immediately anterior to the TMS coil, and held in place by a rubber strap. The cathode was placed medially. Redux gel was used to ensure good contact between the electrodes and the subject's scalp.

Subjects were administered 4 second-trains of real TMS over the prefrontal cortex (10 Hz) at 80%, 100%, and 120% of rMT (randomly ordered) and they rated the painfulness of each sensation using a numeric rating scale (0=no pain at all to 10=worst pain imaginable). These ratings were recorded on the clinical research form for future reference. Next, the sham TMS coil was placed over the subject's prefrontal cortex and the sham system was set to deliver electrical stimuli starting at 1 mA (in sync with the audible TMS pulses at 10 Hz) in trains lasting 4 secs. Subjects were asked to rate the painfulness of each 4-second train using the same numeric rating scale. The intensity of the electrical pulses were adjusted and a PEST algorithm was used to match the subjective pain rating of the electrical stimulation to the rating of the real TMS at 100%. A minimum of 30 secs elapsed between all of the 4-second pulse trains.

### Study Design:

Subjects received a total of 12 4-sec stimulus trains. Half of the trains were delivered using the real TMS coil at 80%, 100% or 120% of rMT (2 trials each). The other 6 trains were delivered using the sham coil and the electrical stimulator at 80%, 100% or 120% (2 trains each) of the mA setting that was matched to real TMS (at 100% of rMT) during the titration process. The order of stimuli was randomized. Subjects were blind to whether the stimuli were real or sham TMS and they were not told the intensity of each stimulus.

### Measuring Pain Location, Quality and Intensity:

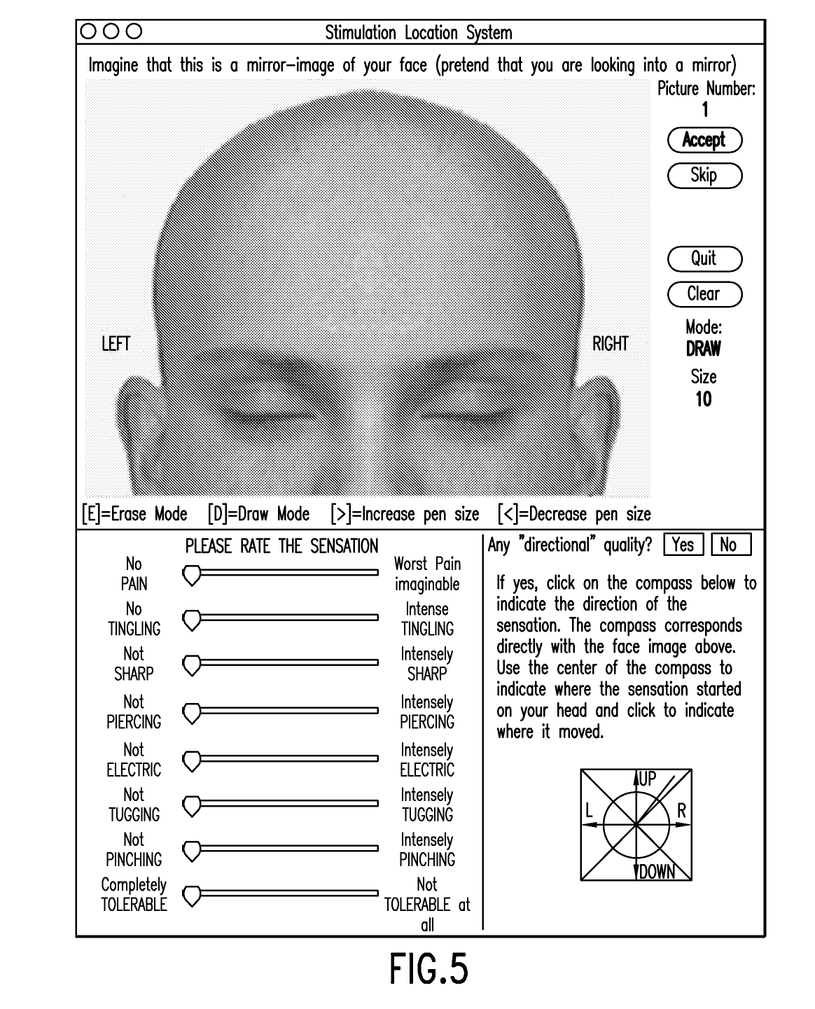

After each stimulus was delivered, subjects used a custom-developed program (by the first author) with several visual analogue scales to rate the sensation (pain, tingling, sharp, piercing, electrical, tugging, pinching, and overall tolerability). They also used the computer mouse to draw on a picture of a human face to indicate where the sensation(s) were felt. Lastly, subjects indicated whether the sensation had a directional quality (i.e., whether it felt like the sensation “moved” across their skin) and, if so, they indicated the direction that the sensation moved using an on screen “compass.” FIG. 5 shows a screen-shot of the custom-developed software.

### Results

Both real and active-sham rTMS were experienced as mildly to moderately painful. Real TMS at 80% of rMT was rated, on average, 19.28 (StdDev=17.52) out of 100 while the sham system was rated as 29.22 (StdDev=25.61). Real TMS at 100% of rMT was rated as 37.06 (StdDev=27.60) and sham TMS was rated on average as 34.61 (StdDev=19.86). Real TMS at 120% rMT was rated as 55.28 (StdDev=31.68) while sham TMS at 120% was rated 39.72 (StdDev=27.56).

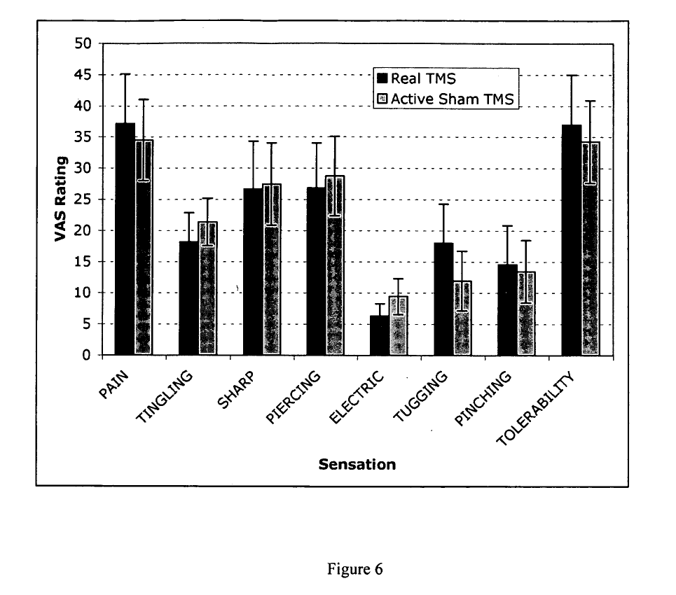

Means (and 95% confidence intervals) for the all of the sensation ratings are shown in FIG. 6 for real and active-sham TMS conditions. No significant differences were found between real and active-sham TMS for any of the sensation dimensions. Break-down of the sensation ratings by stimulus intensity (80%, 100%, 120%) did not reveal any differences on any of the sensation dimensions between real and sham TMS.

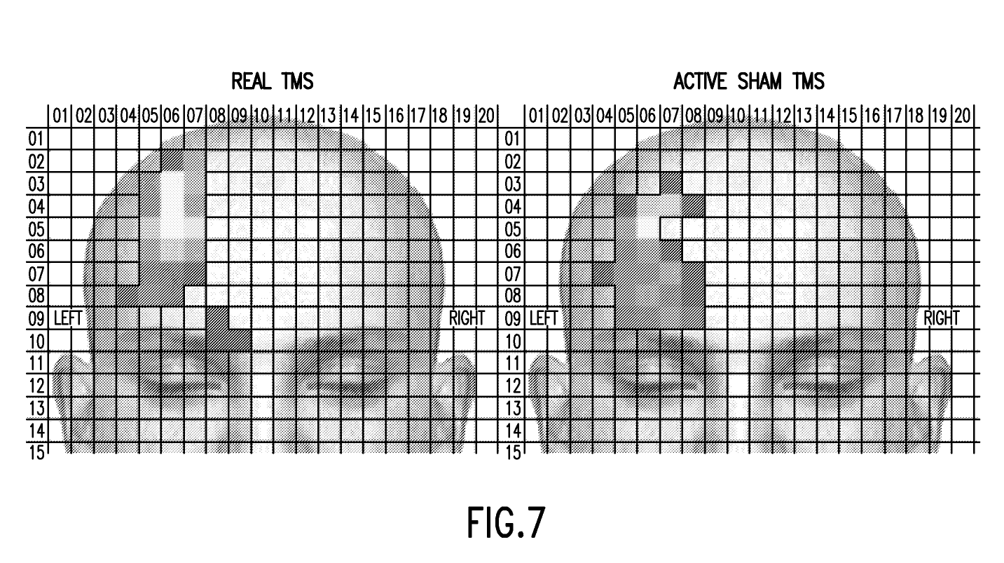

The computerized drawings of the facial/scalp sensation locations were compiled and common areas of activation were determined as the mean number of colored pixels across subjects within 20 by 20 pixel squares. FIG. 7 shows the face and scalp areas that produced sensations under both real and active-sham TMS conditions. The active-sham system produced sensations in the same general facial/scalp areas as real TMS, although the sham system sensations appeared to be experienced slightly lower on the forehead than real TMS.

The sensations were not more likely to be perceived as having a directional quality as a function of the real or sham system and there were no differences in directionality of the sensations between conditions.

### Discussion

The active sham system appears to produce face and scalp sensations that are comparable to real TMS. Additionally, the location of the sensations appear to be comparable between the two conditions (real and sham). The sham system produced sensations slightly lower on the forehead which may be due to the fact that the sham-system electrodes were (by necessity) placed immediately anterior to the TMS coil.

Repetitive TMS over the left-prefrontal cortex appears to be mildly to moderately painful. Typical rTMS clinical and research settings involve repeated stimulation at 100% or 120% of rMT. The average pain intensity ratings of such stimulation in this pilot were 37.06 and 55.28 out of 100, respectively. This degree of pain intensity is substantial enough that it should not be overlooked in future trials of TMS for pain (or for any other disorders or conditions). If sham TMS systems that produce no physical sensations continue to be used, it will continue to be difficult to discern whether any observed anti-nociceptive TMS effects are due to cortical stimulation or are just the result of having subjects undergo a mildly to moderately painful 20-minute procedure.

The sham system employed in this pilot appears to be safe and there were not reports of any side effects. We do not believe that there is any theoretical or empirical evidence to suggest that the electrical stimulation at the levels used in this study (ranging from 2 mA to 7 mA) delivered to the scalp would reach the cortex and result in any unintended cortical or subcortical effects.

The system was built for under $350 and the components sit on top of the TMS machine allowing for good portability. This is important as TMS research continues to expand to into diverse clinical areas, including the post-anesthesia care unit (Borckardt et al, 2006).

TABLE 2
Mean (and standard deviation) painfulness ratings of real
and active-sham TMS at 3 different TMS intensities expressed
as a percentage of resting motor threshold.

| Intensity | Condition | Mean Pain Rating | Std. Deviation |
|---|---|---|---|
| 80% | Real | 19.28 | 17.522 |
| | Sham | 29.22 | 25.607 |
| 100% | Real | 37.06 | 27.603 |
| | Sham | 34.61 | 19.856 |
| 120% | Real | 55.28 | 31.676 |
| | Sham | 39.72 | 27.563 |

### REFERENCES

1. Drouot X, Nguyen J P, Pechanski M, Lefaucheur J P: The antalgic efficacy of chronic motor cortex stimulation is related to sensory changes in the painful zone. Brain 2002; 125:1660-4.
2. Tsubokawa T, Katayama Y, Yamamoto T, Hirayama T, Koyama S: Chronic motor cortex stimulation in patients with thalamic pain. J Neurosurg 1993; 78:393-401
3. Meyerson B A, Lindblom U, Linderoth B, Lind G, Herregodts P: Motor cortex. stimulation as treatment of trigeminal neuropathic pain. Acta Neurochir Suppl (Wein)1993; 58:150-3.
4. Nguyen J P, Keravel Y, Feve A, Uchiyama T, Cesaro P, Le Guerinel C, Pollin B: Treatment of deafferentation pain by chronic stimulation of the motor cortex: Report of a series of 20 cases Acta Neurochir Suppl 1997; 68:54-60.
5. Peyron R, Garcia-Larrea L, Gregoire M C, Convers P, Richard A, Lavenne F, Barral F G, Mauguiere F, Michel D, Laurent B: Parietal and cingulate processes in central pain: A combined positron emission tomography (PET) and functional magnetic resonance imaging (fMRI) study of an unusual case. Pain 2000; 84:77-87.
6. Garcia-Larrea L, Peyron R, Mertens P, Gregoire M C, Lavenne F, Le Bars D, Convers P, Mauguiere F, Sindou M, Laurent B: Electrical stimulation of the motor cortex for pain control: A combined PET-scan and electrophysiological study. Pain 1999; 83:259-73.
7. George M S, Nahas Z, Kozol F A, Li X, Yamanaka K, Mishory A, Bohning D E: Mechanisms and the current state of transcranial magnetic stimulation. CNS Spectr 2003; 8:496-514.
8. Barker A T, Jalinous R, Freeston I L: Non-invasive magnetic stimulation of the human motor cortex. Lancet 1985; 1:1106-7.
9. Barker A T, Freeston I L, Jarratt J A, Jalinous R: Magnetic stimulation of the human nervous system: An introduction and basic principles, Magnetic Stimulation in Clinical Neurophysiology. Edited by Chokroverty S. Boston, Butterworth's, 1989, pp 55-72.
10. George M S, Lisanby S H, Sackeim HA: Transcranial magnetic stimulation: Applications in neuropsychiatry. Arch Gen Psychiatry 1999; 56:300-11.
11. Ziemann U, Hallett M: Basic neurophysiological studies with TMS, Transcranial Magnetic Stimulation in Neuropsychiatry, 1st ed. Edited by George M S, Belmaker R H. Washington, D.C., American Psychiatric Press, 2000, pp 45-98.
12. Roth B J, Saypol J M, Hallett M, Cohen L G: A theoretical calculation of the electric field induced in the cortex during magnetic stimulation. Electroencephalogr Clin Neurophysiol 1991; 81:47-56.
13. Amassian V E, Eberle L, Maccabee P J, Cracco R Q: Modelling magnetic coil excitation of human cerebral cortex with a peripheral nerve immersed in a brain-shaped volume conductor: The significance of fiber bending in excitation. Electroencephalogr Clin Neurophysiol 1992; 85:291-301.
14. Davey K R, Cheng C H, Epstein C M: Prediction of magnetically induced electric fields in biologic tissue. IEEE Trans Biomed Eng 1991; 38:418-22.
15. George M S, Wassermann EM: Rapid-rate transcranial magnetic stimulation and ECT. Convuls Ther 1994; 10:251-3.
16. Migita K, Uozumi T, Arita K, Monden S: Transcranial magnetic coil stimulation of motor cortex in patients with central pain. Neurosurgery 1995; 36:1037-9.
17. Rollnik J D, Wustefeld S, Dauper J, Karst M, Fink M, Kossev A, Dengler R: Repetitive transcranial magnetic stimulation for the treatment of chronic pain: A pilot study. Eur Neurol 2002; 48:6-10.
18. Lefaucheur J-P, Drouot X, Keravel Y, Nguyen J-P: Pain relief induced by repetitive transcranial magnetic stimulation of precentral cortex. Neuroreport 2001; 12:2963-5.
19. Topper R, Hfoltys H, Meister I G, Sparing R, Boroojerdi B: Repetitive transcranial magnetic stimulation of the parietal cortex transiently ameliorates phantom limb pain-like syndrome. Clin Neurophysiol 2003; 114:1521-30.
20. Pleger B, Janssen F, Schwenkreis P, Volker B, Maier C, Tegenthoff M: Repetitive transcranial magnetic stimulation of the motor cortex attenuates pain perception in complex regional pain syndrome type I. Neurosci Lett 2004; 356:87-90.
21. Kanda M, Mima T, Oga T, Matsuhashi M, Toma K, Hara H, Satow T, Nagamine T, Rothwell J C, Shibasaki H: Transcranial magnetic stimulation of the sensorimotor and medial frontal cortex modifies human pain perception. Clin Neurophysiol 2003; 114:860-6.
22. Tamura Y, Okabe S, Ohnishi T, N Saito D, Arai N, Mochio S, Inoue K, Ugawa Y: Effects of 1-Hz repetitive transcranial magnetic stimulation on acute pain induced by capsaicin. Pain 2004; 107:107-15.
23. Reid P, Pridmore S: Improvement in chronic pain with transcranial magnetic stimulation (letter). Aus N Z J Psychiatry 2001; 35:252.
24. Summers J, Johnson S, Pridemore S, Oberoi G: Changes to cold detection and pain thresholds following low and high frequency transcranial magnetic stimulation of the motor cortex. Neurosci Lett 2004; 368:197-200.
25. Lefaucheur J P, Drouot X, Menard-Lefaucher I, Nguyen J P: Neuropathic pain controlled for more than a year by monthly sessions of repetitive transcranial magnetic stimulation of the motor cortex. Neurophys Clin 2004; 34:91-5.
26. Lefaucher J P, Drouot X, Nguyen J P: Interventional neurophysiology for pain control: Duration of pain relief following repetitive transcranial magnetic stimulation of the motor cortex. Neurophys Clin 2001; 31:247-52.
27. Canavero S, Bonicalzi V, Dotta M, Vighetti S, Asteggiano G, Cocito D: Transcranial magnetic cortical stimulation relieves central pain. Stereo Funct Neurosurg 2002; 78:192-6.
28. Khedr E M, Kotb H, Kamel N F, Ahmed M A, Sadek R, Rothwell J C: Longlasting antalgic effects of daily sessions of repetitive transcranial magnetic stimulation in central and peripheral neuropathic pain. J Neurol Neurosurg Psychiatry 2005; 76:833-8.
29. Graff-Guerrero A, Gonzalez-Olivera J, Fresan A, Gomez-Martin D, Mendez-Nunez J C, Pellicer F: Repetitive transcranial magnetic stimulation of dorsolateral prefrontal cortex increases tolerance to human experimental pain. Brain Res Cogn Brain Res 2005; 21:153-60.
30. Pridmore S, Oberoi G, Marcolin M, George M: Transcranial magnetic stimulation and chronic pain: current status. Australas Psychiatry 2005; 13:258-65.
31. Lorenz J, Minoshima S, Casey K L: Keeping pain out of mind: The role of the dorsolateral prefrontal cortex in pain modulation. Brain 2003; 126:1079-91.
32. Greenberg I, Perna F, Kaplan M, Sullivan M A: Behavioral and psychological factors in the assessment and treatment of obesity surgery patients. Obes Res 2005; 13:244-9.
33. George M S, Nahas Z, Li X, Anderson B, Molnar C, Kose S, Borckardt J J, Ricci R, Mu Q: Current status of daily repetitive transcranial magnetic stimulation for the treatment of depression. Primary Psychiatry 2005; 12:51-8.
34. Gershon A A, Dannon P N, Grunhaus L: Transcranial magnetic stimulation in the treatment of depression. Am J Psychiatry 2003; 160:835-45.
35. Awiszus F: TMS and threshold hunting. Suppl Clin Neurophysiol 2003; 56:13-23.
36. Mishory A, Molnar C, Koola J, Li X, Kozel F A, Myrick H, Stroud Z, Nahas Z, George M S: The maximum-likelihood strategy for determining transcranial magnetic stimulation motor threshold, using parameter estimation by sequential testing is faster than conventional methods with similar precision. J ECT 2004; 20:160-5.
37. Wassermann EM: Risk and safety of repetitive transcranial magnetic stimulation: Report and suggested guidelines from the International Workshop in the Safety of Repetitive Transcranial Magnetic Stimulation, Jun. 5-7, 1996. Electroencephalogr Clin Neurophysiol 1998; 108:1-16.
38. Pridmore S, Fernandes Filho J A, Nahas Z, Liberatos C, George M S: Motor threshold in transcranial magnetic stimulation: A comparison of a neurophysiological method and a visualization of movement method. J ECT 1998; 14:25-7.
39. Bryk A S, Raudenbush S W: Application of hierarchical linear models to assessing change. Psychol Bull 1987; 101:147-58.
40. Bryk A S, Raudenbush S W: Hierarchical Linear Models: Applications and Data Analysis Methods. Newbury Park, Calif., Sage Publications, 1992.
41. Banks S, Kerns R: Explaining high rates of depression in chronic pain: A diathesis-stress framework. Psychol Bull 1996; 119 95-110.

We claim:

1. A device for delivering a sham TMS sensation comprising:
(a) an electrical stimulation generator;
(b) one or more electrodes capable of being affixed to a subject, said electrodes being electrically connected to the electrical stimulation generator;
(c) a means for interfacing the electrical stimulation generator to a TMS machine;
wherein the electrical stimulation generator generates an electrical pulse delivered to the subject via the one or more electrodes at substantially the same time the TMS machine generates a sham TMS pulse.

2. A method for the treatment of acute pain comprising delivering repetitive transcranial magnetic stimulation (rTMS) for about 20 minutes to the left prefrontal cortex of a subject in need of treatment, wherein the rTMS parameters comprise about 10 Hz, about 100% rMT, about 10 seconds ON, and about 20 seconds OFF.
